(19) United States
(12) Patent Application Publication
(10) Pub. No.: US 2004/0170657 A1
(43) Pub. Date: Sep. 2, 2004

(54) Dispersion comprising an emulsion having an aqueous phase with high ionic strength

(76) Inventors: Mikel Morvan, Princeton, NJ (US); Alain Senechal, Charenton (FR)

Correspondence Address: Jean-Louis Seugnet, Rhodia Inc, Intellectual Property Dept., 259 Prospect Plains Road CN 7500, Cranbury, NJ, 08512-7500, US

(21) Appl. No.: 10/481,045

(22) PCT Filed: Jun. 27, 2002
(86) PCT No.: PCT/FR02/02239

(30) Foreign Application Priority Data
Jun. 27, 2001 (FR) 01/08744

(51) Int. Cl. A61K 7/00

(52) U.S. Cl. 424/401

(57) ABSTRACT

The invention relates to a dispersion comprising at least one emulsion of an organic phase in an aqueous phase, said aqueous phase comprising a soluble salt content of at least 0.5 mol/l. The invention is characterized in that the aqueous phase contains at least one amphiphilic block copolymer, comprising at least one hydrophilic block, selected from copolymers which: (i) do not display macroscopic separation of phases in solution at 1% weight, in a mixture of water and KBr, the concentration of KBr being identical to that of soluble salt in the dispersion, at 20° C.; (ii) have a hydrophilic block(s)/hydrophobic block(s) mass ratio of between 40/60 and 95/5. The invention also relates to a method of producing said dispersion and phytosanitary formulations.

The present invention relates to dispersions comprising an oil-in-water emulsion, such that the ionic strength of the aqueous phase of the emulsion is high, as well as to a method of preparation of said dispersions and their uses in the areas of plant protection formulations.

Dispersions, and notably emulsions, are being used increasingly for combining active substances that are not miscible, or are even incompatible, with one another. In this scenario; a case that is often encountered is when it is necessary to stabilize, in the form of an emulsion, a hydrophobic active substance and a hydrophilic active substance in the form of water-soluble salt. This form of application does not present much of a problem when the dispersions are such that the aqueous phase is of relatively low ionic strength. However, the situation is quite different when the concentration of active substance(s) becomes very high in said phase, in other words when the ionic strength of the aqueous phase is high. Now, it is important to be able to have dispersions where there is a high concentration of active substance(s), so as to increase their efficacy (higher content, mixture of active substances with optimization of the proportions, among other things). These dispersions are then intended to be diluted by the user, prior to their application.

The difficulty is that when preparing emulsions having high ionic strength of the aqueous phase, most of the surfactants used conventionally for stabilizing emulsions can no longer be used, since they become insoluble in such media. There are some surfactants that are able to stabilize emulsions of this type, but their number is relatively limited, and they belong inter alia to the family of surfactants with a sugar head group, for example the alkylpolyglucosides. Even if these surfactants are able to stabilize emulsions of high ionic strength, their use is not completely satisfactory. Thus, during dilution of the emulsions by the user, regions of instability appear (flocculation, coalescence). As a result, the application of these diluted emulsions is still unsuitable.

The aim of the present invention is to solve the problems mentioned above.

Thus, one of the first objects of the invention therefore consists of a dispersion comprising at least one emulsion of an organic phase in an aqueous phase; the aqueous phase having a content of soluble salt of at least 0.5 mol/l, characterized in that the aqueous phase contains at least one amphiphilic block copolymer, comprising at least one hydrophilic block, selected from the copolymers:

(i) which do not cause macroscopic phase separation in solution at 1 wt. %, in a mixture of water and KBr; the concentration of KBr being identical to that of soluble salt of the dispersion, at 20° C.;

(ii) whose mass ratio of hydrophilic block(s) to hydrophobic block(s) is between 40/60 and 95/5.

A second object of the invention consists of a method of production of a dispersion, consisting of preparing an aqueous solution of copolymer and of soluble salt, to which the organic phase, possibly containing an active substance, is added.

Another object of the invention relates to the use of said dispersions in the areas of plant protection formulations.

The present invention provides a satisfactory solution for the problem of stabilization of emulsion of high ionic strength. In fact, it is possible to obtain stable concentrated dispersions, notably emulsions.

Moreover, the dispersions according to the invention conserve this property of stability when they are diluted, prior to their application.

However, other advantages and characteristics of the present invention will become clearer on reading the description and the examples given below.

In the sense of the present invention, the word “dispersion” means, depending on circumstances, dispersions of several liquids that are at least partly immiscible (emulsions) or dispersions of several liquids that are at least partly immiscible and of solid particles (suspo-emulsions).

As already stated above, the invention relates to a dispersion comprising at least one emulsion of an organic phase in an aqueous phase having a content of soluble salt of at least 0.5 mol/l and containing at least one amphiphilic block copolymer selected from the copolymers:

(i) which do not cause macroscopic phase separation in solution at 1 wt. %, in a mixture of water and KBr with a concentration of KBr which is identical to that of soluble salt of the dispersion, at 20° C.;

(ii) whose mass ratio of hydrophilic block(s) to hydrophobic block(s) is between 40/60 and 95/5.

According to one embodiment of the invention, the copolymer employed as stabilizer of the emulsion is selected from those whose mass ratio of hydrophilic block(s) to hydrophobic block(s) is between 50/50 and 90/10.

More particularly, the hydrophilic block or blocks of the copolymer can be obtained by employing, as hydrophilic monomer, at least one ionic or potentially ionizable hydrophilic monomer, more particularly anionic or cationic, and/or at least one nonionic hydrophilic monomer. It should be pointed out that since the blocks of the copolymer possess ionizable functions, they may be in an ionic or nonionic form, depending on the pH of the dispersion and the pK of said functions.

Preferably, the pH of the dispersion is such that the functions of the copolymer are in an at least partially ionized form, or even completely ionized form.

Among the anionic monomers that can be employed for obtaining the hydrophilic block or blocks, we may mention those containing at least one carboxylic, sulfonic, sulfuric, phosphonic, phosphoric, or sulfosuccinic function, or the corresponding salts.

More particularly, the anionic hydrophilic block or blocks of the copolymer can be obtained from at least one monomer selected from:

the linear, branched, cyclic or aromatic mono- or polycarboxylic acids, the N-substituted derivatives of said acids; the monoesters of polycarboxylic acids, containing at least one ethylenic unsatutation;

the linear, branched, cyclic or aromatic vinylcarboxylic acids;

the amino acids containing one or more ethylenic unsaturations;

individually or mixed, their precursors, their sulfonic or phosphonic derivatives, as well as the macromonomers derived from said monomers; it being possible for the monomers or macromonomers to be in the form of salts. Note that the term macromonomer denotes a macromolecule carrying one or more polymerizable functions.

As examples of monomers that can be involved in the preparation of the anionic hydrophilic block or blocks of the copolymer, we may mention, without any intention of being limited thereto:

acrylic acid, methacrylic acid, fumaric acid, itaconic acid, citraconic acid, maleic acid, acrylamido glycolic acid, 2-propene-1-sulfonic acid, methallyl sulfonic acid, styrene sulfonic acid, α-acrylamido-methylpropane sulfonic acid, 2-sulfoethylene methacrylate, sulfopropyl acrylic acid, bis-sulfopropyl acrylic acid, bis-sulfopropyl methacrylic acid, sulfatoethyl methacrylic acid, the phosphate monoester of hydroxyethyl methacrylic acid, as well as the salts of alkali metal, such as sodium, potassium, or of ammonium;

vinyl sulfonic acid, vinylbenzene sulfonic acid, vinyl phosphonic acid, vinylidene phosphoric acid, vinyl benzoic acid, as well as the salts of alkali metal, such as sodium, potassium, or of ammonium;

N-methacryloyl alanine, N-acryloyl-hydroxy-glycine; individually or mixed, as well as the macromonomers derived from said monomers.

It should be noted, as stated previously, that the use of monomers that are precursors of those that have just been mentioned is also within the scope of the present invention. In other words, these monomers have units which, once incorporated in the polymer chain, can be transformed, notably by a chemical treatment such as hydrolysis, to give the aforementioned anionic species again. For example, the fully or partially esterified monomers of the aforementioned monomers can be used, and can then be hydrolyzed, completely or partially.

Regarding the cationic monomers that can be included in the composition of the hydrophilic block or blocks, we may mention among others:

the aminoalkyl (meth)acrylates, the aminoalkyl (meth)acrylamides;

monomers containing at least one secondary, tertiary or quaternary amine function, or a heterocyclic group containing a nitrogen atom, vinylamine, ethyleneimine;

diallyldialkylammonium salts;

individually or mixed, or the corresponding salts; as well as the macromonomers derived from said monomers.

Thus, when the monomers are in the form of salts (more particularly with quaternized amine functions), of ammonium type NR₄+ with R, identical or not, representing a hydrogen atom, an alkyl radical containing 1 to 10 carbon atoms, preferably 1 to 4, possibly bearing a hydroxyl radical, the counterion can be selected from the halides for example chlorine, the sulfates, hydrosulfates, alkylsulfates (for example containing 1 to 6 carbon atoms), the phosphates, citrates, formates, or acetates.

Examples of suitable cationic monomers include the following monomers:

dimethyl aminoethyl (meth)acrylate, dimethyl aminopropyl (meth)acrylate, ditert-butyl aminoethyl (meth)acrylate, dimethyl aminomethyl (meth)acrylamide, dimethyl aminopropyl (meth)acrylamide;

ethylene imine, vinylamine, 2-vinylpyridine, 4-vinylpyridine;

the chloride of trimethylammonium ethyl (meth)acrylate, the methyl sulfate of trimethylammonium ethyl acrylate, the chloride of benzyl dimethylammonium ethyl (meth)acrylate, the chloride of 4-benzoylbenzyl dimethyl ammonium ethyl acrylate, the chloride of trimethyl ammonium ethyl (meth)acrylamide, the chloride of vinylbenzyl trimethyl ammonium;

the chloride of diallyldimethyl ammonium;

individually or mixed, or their corresponding salts, as well as the macromonomers derived from said monomers.

Regarding the nonionic hydrophilic monomers that can be used for the preparation of hydrophilic blocks, we may mention among others ethylene oxide; the amides of linear, branched, cyclic or aromatic mono- or polycarboxylic acids, containing at least one ethylenic unsaturation or derivatives, such as (meth)acrylamide, N-methylol(meth)acrylamide; cyclic amides of vinylamine, such as N-vinylpyrrolidone; the hydrophilic esters derived from (meth)acrylic acid, for example 2-hydroxyethyl (meth)acrylate; vinyl esters by means of which it is possible to obtain polyvinyl alcohol blocks after hydrolysis, such as vinyl acetate, vinyl Versatate®, vinyl propionate; N-vinyl monomers such as N-vinylcaprolactone, N-vinylcaprolactam, N-vinylacetamide; ethylenic monomers containing a ureido group such as ethylene urea ethyl (meth)acrylamide, or ethylene urea ethyl (meth)acrylate; monomers of the sugar type such as osides, highly depolymerized polyholosides, individually, in combination, as well as the macromonomers derived from said monomers.

The term highly depolymerized polyholosides means polyholosides whose weight-based molecular mass is more particularly less than 20000 g/mol.

The osides are compounds that result from the condensation, with elimination of water, of molecules of oses with one another or of molecules of oses with non-glucidic molecules. Among the osides, the holosides are preferred, which are formed by joining together exclusively glucidic units and more particularly the oligoholosides (or oligosaccharides) which only have a limited number of these units, i.e. a number generally less than or equal to 10. As examples of oligoholosides we may mention sucrose, lactose, cellobiose, and maltose.

The suitable highly depolymerized polyholosides (or polysaccharides) are described for example in the work by P. Arnaud with the title “Course of Organic Chemistry”, publ. Gauthier-Villars, 1987. As non-limiting examples of highly depolymerized polyholosides we may mention dextran and starch.

Said monomers can be used individually or as mixtures, as well as in the form of macromonomers.

However, the preferred, ionic or nonionic hydrophilic monomers included in the compositions of the hydrophilic blocks of the copolymers are more particularly selected from acrylic acid or its salts, acrylamide, 2-acrylamido-2-methylpropanesulfonic acid or its salts, and styrenesulfonic acid or its salts, vinylbenzyl trimethylammonium chloride, individually or in mixtures, or in the form of macromonomers.

As illustration of hydrophobic monomers that can be used for preparing the hydrophobic block or blocks, we may mention notably:

the linear, branched, cyclic or aromatic esters of mono- or polycarboxylic acids, containing at least one ethylenic unsaturation,

αβ-ethylenically unsaturated nitrites, vinyl ethers, vinyl esters, vinylaromatic monomers, halides of vinyl or of vinylidene,

linear or branched, aromatic or non-aromatic hydrocarbon monomers, containing at least one ethylenic unsaturation,

individually or mixed, as well as the macromonomers derived from said monomers.

As particular examples of monomers that can be used in the preparation of the hydrophobic block or blocks of the copolymers, we may mention:

the esters of (meth)acrylic acid with an alcohol containing 1-12 carbon atoms such as methyl (meth)acrylate, ethyl (meth)acrylate, propyl (meth)acrylate, n-butyl (meth)acrylate, t-butyl (meth)acrylate, isobutyl (meth)acrylate, 2-ethylhexyl acrylate;

vinyl acetate, vinyl Versatate®, vinyl propionate, vinyl chloride, vinylidene chloride, methyl vinyl ether, ethyl vinyl ether;

vinyl nitrites including more particularly those having from 3 to 12 carbon atoms, such as in particular acrylonitrile and methacrylonitrile;

styrene, α-methylstyrene, vinyltoluene, paramethylstyrene, paratert-butyl styrene, butadiene, chloroprene, isoprene;

individually or mixed, as well as the macromonomers derived from said monomers.

The monomers that are preferred are the esters of acrylic acid with linear or branched C₁-C₄ alcohols such as methyl, ethyl, propyl and butyl acrylate, the vinyl esters such as vinyl acetate, styrene, α-methylstyrene.

It should be noted that at least one of the hydrophilic blocks can be made up of one or more hydrophilic monomers or alternatively of a mixture of hydrophilic and hydrophobic monomer(s); the proportion by weight of hydrophobic monomer(s) representing more particularly less than 50% of the monomers forming said hydrophilic block, and preferably less than 20%. In the case of a mixture of monomers of different types, the monomers are more particularly combined either in a mixture of nonionic and/or anionic hydrophilic monomers and of hydrophobic monomers, or of nonionic and/or cationic hydrophilic monomers and of hydrophobic monomers. It should also be noted that when the blocks include a mixture of monomers, the latter are distributed randomly within said block.

The same remark can be made for the hydrophobic blocks.

Thus, at least one of the hydrophobic blocks can be made up of one or more hydrophobic monomers or alternatively of a mixture of hydrophobic and hydrophilic monomer(s); the proportion-by mass of hydrophilic monomer(s) representing more particularly less than 50 wt. % of the monomers forming said hydrophobic block. Once again, in the case of a mixture of monomers of different types, the monomers are more particularly combined either in a mixture of nonionic and/or anionic hydrophilic monomers and of hydrophobic monomers, or of nonionic and/or cationic hydrophilic monomers and of hydrophobic monomers. Finally, when the blocks include a mixture of monomers, the latter are distributed randomly within said block.

Moreover, more particularly, the copolymer is such that the number-based molar mass of each block is between 500 and 100·10³ g/mol, preferably between 10³ and 50·10³ g/mol.

Note that the weight-based molar masses indicated above are theoretical molar masses, evaluated in relation to the respective quantities of the monomers and of the control agent introduced during preparation of said polymers.

The copolymers that can be used as stabilizer are advantageously copolymers of linear form.

The copolymers employed are block copolymers, and preferably diblocks.

These copolymers are compounds that are familiar to a person skilled in the art and can be obtained by ionic polymerization or, preferably, so-called living or controlled radical polymerization involving the use of a control agent for controlling said radical polymerization.

The use of living radical polymerization offers the advantage of controlling the composition of each of the blocks as well as their stereoregularity (in the case of blocks containing monomers of different types).

Regarding the methods of polymerization called living or controlled, reference may be made notably to:

the methods of applications WO 98/58974, WO 00/75207 and WO 01/42312 which employ a radical polymerization controlled by control agents of xanthate type,

the method of radical polymerization controlled by control agents of dithioester type according to application WO 98/01478,

the method of application WO 99/03894 that employs polymerization in the presence of nitroxide precursors,

the method of radical polymerization controlled by control agents of dithiocarbamate type according to application WO 99/31144,

the method of radical polymerization controlled by control agents of dithiocarbazate type according to application WO 02/26836,

the method of radical polymerization controlled by control agents of dithiophosphoroester type according to application WO 02/10223,

the method of application WO 96/30421 that uses atom transfer radical polymerization (ATRP),

the method of radical polymerization controlled by control agents of iniferter type according to the teaching of Otu et al., Makromol. Chem. Rapid Commun., 3, 127 (1982),

the method of radical polymerization controlled by degenerative transfer of iodine according to the teaching of Tatemoto et al., Jap. 50, 127, 991 (1975), Daikin Kogyo Co. Ltd. Japan and Matyjaszewski et al., Macromolecules, 28, 2093 (1995),

the method of radical polymerization controlled by tetraphenylethane derivatives, disclosed by D. Braun et al. in Macromol. Symp. 111, 63 (1996), or

the method of radical polymerization controlled by organocobalt complexes described by Wayland et al. in J. Am. Chem. Soc. 116, 7973 (1994).

The preferred control agents for applying the method of controlled polymerization are selected from the dithioesters, dithiocarbamates, and better still, from the xanthates.

The method comprises carrying out the following:

(a) in a first step, at least one ethylenically unsaturated monomer, at least one source of free radicals, and at least one control agent are brought into contact, then

(b) in a second step, at least one ethylenically unsaturated monomer different from that or those used in the preceding step, is brought into contact with the polymer resulting from step (a).

According to a particularly advantageous embodiment, the control agent of step (a) corresponds to the following formula:

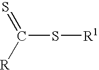

in which:

R represents a group R²O—, R²R′²N— or R³— with:

R² and R′², identical or different, representing (i) an alkyl, acyl, aryl, alkene or alkyne group, or (ii) a carbon ring, saturated or unsaturated, possibly aromatic, or (iii) a heterocycle, saturated or unsaturated, said groups and rings (i), (ii) and (iii) possibly being substituted,

R³ representing H, Cl, an alkyl, aryl, alkene or alkyne group, a saturated or unsaturated (hetero)cycle, possibly substituted, an alkylthio, alkoxycarbonyl, aryloxycarbonyl, carboxy, acyloxy, carbamoyl, cyano, dialkyl- or diaryl-phosphonato, dialkyl- or diaryl-phosphinato group, a polymer chain,

R¹ represents (i) an alkyl, acyl, aryl, alkene or alkyne group, possibly substituted, or (ii) a carbon ring, saturated or unsaturated, possibly substituted or aromatic, or (iii) a heterocycle, saturated or unsaturated, possibly substituted, or a polymer chain. The groups R¹, R², R¹² and R³ can be substituted by alkyl groups, substituted phenyl groups, substituted aromatic groups or the groups: oxo, alkoxycarbonyl or aryloxycarbonyl (—COOR), carboxy (—COOH), acyloxy (—O₂CR), carbamoyl (—CONR₂), cyano (—CN), alkylcarbonyl, alkarylcarbonyl, arylcarbonyl, aralkylcarbonyl, isocyanato, phthalimido, maleimido, succinimido, amidino, guanidino, hydroxy (—OH), amino (—NR2), halogen, allyl, epoxy, alkoxy (—OR), S-alkyl, S-aryl, silyl, groups having a hydrophilic or ionic character such as the alkaline salts of carboxylic acids, the alkaline salts of sulfonic acid, alkylene polyoxide chains (POE, POP), cationic substituents (quaternary ammonium salts), R representing an alkyl or aryl group.

Preferably, the control agent of formula (I) is a xanthate selected from the compounds of the following formulae (A), (B) and (C):

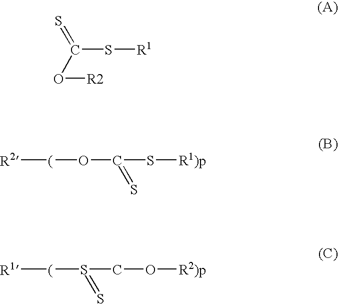

in which:

R² and R²¹ represent (i) an alkyl, acyl, aryl, alkene or alkyne group, or (ii) a carbon ring, saturated or unsaturated, possibly aromatic, or (iii) a heterocycle, saturated or unsaturated, said groups and rings (i), (ii) and (iii) possibly being substituted,

R¹ and R¹′ represent (i) an alkyl, acyl, aryl, alkene or alkyne group, possibly substituted, or (ii) a carbon ring, saturated or unsaturated, possibly substituted or aromatic, or (iii) a heterocycle, saturated or unsaturated, possibly substituted, a polymer chain,

p is between 2 and 10.

During step (a), a first block of the polymer is synthesized hydrophilically or hydrophobically depending on the nature and the quantity of the monomers used. During step (b), the other block of the polymer is synthesized.

The monomers are used in suitable proportions to obtain a block copolymer that has the characteristics mentioned previously, i.e. at least of being soluble in aqueous media of high ionic strength, as well as the mass ratio of the hydrophilic/hydrophobic block(s).

It should be noted that if the successive polymerizations are carried out in the same reactor, it is generally preferable that all the monomers used during one step have been consumed before the polymerization of the next step begins, therefore before the new monomers are introduced. However, it may happen that the hydrophobic or hydrophilic monomers of the preceding step are still present in the reactor during the polymerization of the next block. In this case, said monomers do not generally represent more than 5 mol. % of all the monomers and they take part in the subsequent polymerization by contributing to the introduction of hydrophobic or hydrophilic units to the next block.

Advantageously, the polymerization can be carried out in an aqueous medium and/or organic solvent such as tetrahydrofuran or a linear, cyclic or branched C₁-C₈ aliphatic alcohol such as methanol, ethanol, or cyclohexanol, or a diol such as ethyleneglycol. An alcohol, or a water/alcohol mixture is recommended more particularly in the case when the hydrophilic monomers are acrylic acid, acrylamide, 2-acrylamido-2-methylpropanesulfonic acid, and the styrene sulfonate and the hydrophobic monomers are n-butyl acrylate, isobutyl acrylate, 2-ethylhexyl acrylate, or t-butyl acrylate.

At the end of the step of controlled polymerization, the control agent, located at one end of the polymer chain, can be made inert if desired for the ultimate use of the copolymer. It is possible that the nature of the reaction medium of polymerization (for example conditions of pH, nature of the constituents of the reaction medium, monomers to be polymerized), is sufficient in itself to inactivate the control agent at the end of polymerization. It is also possible that the medium to be treated during the ultimate use of the copolymer inactivates or neutralizes the control agent inherently. If it is necessary for certain applications, it is recommended to mask the active chemical functions of said agent by means of a suitable chemical masking agent, or to destroy the control agent by a reaction of hydrolysis, or oxidation by metallic catalysis. In the case of xanthate as control agent, it is recommended to make it inert, if necessary, by treating the copolymer that has formed by means of a heat treatment, for example in the temperature range 80 to 180° C. in the presence of an alkanolamine such as triethanolamine.

The kind of preparation that has just been detailed, as well as the resulting copolymers, were described in international application PCT/FR02/01349 of Apr. 19, 2002.

The content of copolymer in the dispersion according to the invention represents more particularly at least 1 wt. % relative to the organic phase, advantageously from 2 to 20 wt. % relative to the organic phase, and preferably between 2 and 10 wt. % relative to the organic phase.

As already described, the aqueous phase of the dispersion includes at least one water-soluble salt whose concentration is at least 0.5 mol/l.

According to a particular embodiment of the invention, the concentration of salt soluble in the aqueous phase is greater than 0.8 mol/l; advantageously greater than or equal to 1 mol/l, more particularly greater than or equal to 1.7 mol/l, and preferably greater than or equal to 2 mol/l.

Any organic or mineral salt can be included in the composition of the dispersion as soon as it forms, at 20° C., a solution in water, with a concentration within the ranges mentioned above.

According to a preferred embodiment of the invention, said soluble salts are active substances in the area of application of the dispersion according to the invention.

Thus, in the area of plant protection formulations, we may mention, among others, hydrophilic pesticides, and more especially herbicides, but also the hydrophilic nutrients promoting plant growth and development.

Among the suitable active substances, we may mention especially the following herbicidal active substances in the form of organic or inorganic salts: the aminophosphate or aminophosphonate derivatives, Acifluorfen, Asulam, Bentazon, Bialaphos, Bispyribac, Bromacil, Bromoxynil, Chloramben, Clopyralid, 2,4-D, 2,4-Db, Dalapon, Dicamba, Dichlorprop, Difenzoquat, Diquat, Endothall, Fenac, Fomesafen, Fosamine, Ioxynil, MCPA, MCPB, Mecoprop, Methylarsonic Acid, Naptalam, Paraquat, Picloram, Sulfamic Acid, individually or mixed.

Combining one or more of the aforementioned active substances still falls within the scope of the present invention.

The active substance is preferably selected from the aminophosphate or aminophosphonate derivatives, in the form of organic or inorganic salts such as glyphosate, sulfosate, glufosinate, in the form of organic or inorganic salts.

Glyphosate denotes more particularly N-phosphonomethylglycine as well as any derivative of the latter leading to glyphosate anions in aqueous solution.

As suitable salts, we may mention more particularly the salts of alkali metals such as sodium or potassium; the salts of ammonium, of type N(R)₄+ for which the R radicals, identical or different, represent a hydrogen atom or a linear or non-linear, saturated or unsaturated C₁-C₆ hydrocarbon radical, possibly substituted by a hydroxyl group; or alternatively sulfonium salts; said salts being present individually or in combination.

Among the ammonium salts, we may mention quite particularly the secondary or primary amines such as isopropylamine, dimethylamine or the diamines such as ethylenediamine. As for the sulfonium salts, trimethylsulfonium is perfectly suitable.

As preferred derivatives of glyphosate, we may mention notably the salt of isopropylamine and the salt of trimethylsulfonium.

With regard to the nutrients, these are preferably metal salts such as salts of zinc, of iron, and preferably of manganese. These salts are used in the form of chelates for example of EDTA type, or of sulfates. In the case when the nutrients are polyvalent metals, it is preferable that the hydrophilic blocks of the copolymer are not obtained from cationic monomers.

According to one variant of the present invention, the dispersion can comprise a solid dispersed in the aqueous phase of the latter.

Advantageously, if such a solid is present, it is selected from the active compounds.

Among the solid active substances that can be included in the composition of the dispersion according to the invention, we may mention, among others, the compounds of the triazine family, such as atrazine, ametryn, cyanazine, simazine, terbuthylazine, or their mixtures.

Furthermore, preferably, the number-average size of the solid particles is between 0.3 and 10 μm, advantageously between 0.5 and 2 μm.

The organic phase of the emulsion is selected from the organic compounds that are in a liquid form at the temperature of preparation of the dispersion according to the invention, or alternatively from those that are liquid at the temperature of use of said dispersion, or even these two possibilities simultaneously.

As examples of suitable organic phases for preparation of the dispersion according to the invention, we may mention for example organic oils, waxes or fats of vegetable or animal origin; mineral oils or waxes; saturated or unsaturated fatty acids; esters of said acids; saturated or unsaturated fatty alcohols; aliphatic or aromatic hydrocarbon solvents; petroleum fractions; individually or mixed.

As organic oils of animal origin we may mention, among others, sperm oil, whale oil, seal oil, sardine oil, herring oil, dogfish oil, cod-liver oil; pork fat (lard), mutton fat (suet).

As waxes of animal origin we may mention beeswax.

As examples of organic oils of vegetable origin we may mention, among others, colza oil, sunflower oil, peanut oil, olive oil, walnut oil, corn oil, soybean oil, linseed oil, hemp oil, grape pip oil, coconut oil, palm oil, cotton-seed oil, jojoba oil, sesame oil, castor oil.

As waxes of vegetable origin, we may mention carnauba wax.

As for the mineral oils, we may mention among others the naphthenic oils and paraffin oils (vaseline). Paraffin and isoparaffin waxes may also be suitable as the organic phase of the emulsion.

Products obtained from alcoholysis of the aforementioned oils can also be used.

We shall remain within the scope of the present invention if we use, as organic phase, at least one saturated or unsaturated fatty acid, at least one saturated or unsaturated ester of fatty acid, and of alcohol containing 1 to 6 carbon atoms, at least one saturated or unsaturated fatty alcohol, or their mixtures.

More particularly, said acids, esters or alcohols contain 10 to 40 carbon atoms, more particularly 18 to 40 carbon atoms, and can contain one or more ethylenic unsaturations, conjugated or unconjugated. Moreover, the acids, esters or alcohols can contain one or more hydroxyl groups.

As examples of saturated fatty acids, we may mention palmitic, stearic and behenic acids.

As examples of unsaturated fatty acids, we may mention myristoleic, palmitoleic, oleic, erucic, linoleic, linolenic, arachidonic, ricinoleic acids, as well as their mixtures.

As esters of fatty acids, we may mention the esters of the acids listed previously, for which the part derived from the alcohol has 1 to 6 carbon atoms, such as the methyl, ethyl, propyl, isopropyl esters etc.

As examples of alcohols, we may mention those corresponding to the aforementioned acids.

The organic phase can also be selected from the mono-, di- and triglycerides.

Among the aliphatic or aromatic hydrocarbon solvents; the petroleum fractions, we may mention xylene, Solvesso®.

The organic phase can contain one or more active substances, or can even constitute an active substance itself.

Furthermore, the active substance can be in a form that is soluble in the organic phase or alternatively can be dissolved in said phase.

In this case, a triple solvent is present, selected from the compounds that dissolve the active substance but also from those soluble in the organic phase of the dispersion.

As examples of solvents of this type, we may mention the ketones, such as cyclohexanone, isophorone, the lactones, such as butyrolactone, the pyrrolidones, such as N-vinylpyrrolidone, said solvents being alone or in a mixture.

Note that the content of said triple solvents in the dispersion remains such that the active substance that is soluble in the organic phase remains soluble in the organic phase and not in the aqueous phase of the dispersion.

As examples of active substances that are soluble or are dissolved in the organic phase, and that can be used in the area of plant protection formulations, we may mention for example the active substances selected from the family of the α-cyano-phenoxybenzyl carboxylates or of the α-cyano-halogenphenoxycarboxylates, the family of the N-methylcarbonates containing aromatic substituents, the active substances such as Azinphos-methyl, Benfluralin, Bifenthrin, Chlorphoxim, Chlorpyrifos, Fluchloralin, Fluroxypyr, Dichloruos, Malathion, Molinate, Parathion, Permethrin, Profenofos, Propiconazole, Prothiofos, Pyrifenox, Butachlor, Metolachlor, Chlorimephos, Diazinon, Fluazifop-P-butyl, Heptopargil, Mecarbam, Propargite, Prosulfocarb, Bromophos-ethyl, Carbophenothion, Cyhalothrin, Imazameth, Imazamethabenz, Imazamox, Imazapic, Imazapyr, Imazaquin, Amzethapyr, or their mixtures.

According to a particular embodiment of the invention, the proportion by weight of organic phase in the dispersion represents 5 to 50 wt. %, preferably 10 to 30 wt. %.

According to a variant of the invention, the dispersion can contain at least one surfactant. Preferably, the surfactant or surfactants are selected from those that are soluble in the aqueous phase exhibiting a high ionic strength, i.e. an ionic strength corresponding to a concentration of soluble salt of at least 0.5 mol/l, more particularly greater than 0.8 mol/l, preferably greater than or equal to 1 mol/l and, even more preferred, greater than or equal to 2 mol/l.

Said surfactant can be selected from the anionic or nonionic surfactants.

According to one embodiment of the present invention, the surfactant is selected from the alkylpolyglucosides.

These surfactants are well known to a person skilled in the art. More precisely they are nonionic, oligomeric surfactants, which are in the form of acetals of fatty alcohols (preferably C₈-C₁₆) and of sugars (glucose). Notably they are obtained by chemical reaction from starch and from fats. Such surfactants are for example marketed by the company Henkel under the names Plantaren®.

In fact it was noticed, quite unexpectedly, that there is a synergy between the surfactants of this type with the copolymers that are included in the composition of the dispersions according to the invention.

Thus, when the content of alkylpolyglucoside surfactant represents at most 50 wt. % of the combined weight of surfactant+copolymer, it has been found on the one hand that it is possible to obtain stable emulsions not only in the concentrated state but also after dilution. Now, all things being equal, this is not possible with the alkylpolyglucosides alone.

Moreover, it was noticed that by employing the same means for the preparation of the dispersion, more particularly the emulsion, and notably the same energy means, an emulsion was obtained in which the average droplet size was smaller than that of emulsions obtained starting from one or other of said compounds. Consequently, this leads to a further improvement in the stability of the emulsion.

The dispersion according to the invention (emulsion) has more particularly an average droplet size between 0.3 and 10 μm, preferably between 0.5 and 2 μm (expressed relative to the volume of particles; measured by means of a laser diffraction granulometer of the HORIBA type).

The dispersion according to the invention can in addition contain additives conventionally used in this field.

Thus, in some cases it may be advantageous to add a biological activator to the dispersion. More particularly, one of the roles of this type of additive is to increase the efficacy of some of the active substances contained in the dispersion.

Preferably, the biological activators are present in the aqueous phase of the dispersion and are intended to activate the efficacy of the active substance or substances in the form of water-soluble salt.

A person skilled in the art knows for which active substances it may be preferable to add these biological activators, as well as the nature of the latter. However, as examples of biological activators, in the particular case of the aminophosphates, and quite especially of glyphosate, we may mention notably the polyalkoxylated and notably polyethoxylated fatty amines; the polyalkoxylated (for example polyethoxylated or polyethoxylated/polypropoxylated) amidoamines. Others that are suitable are the polyalkoxylated (for example polyethoxylated or polyethoxylated/polypropoxylated) phosphate mono- and diesters of linear or non-linear, saturated or unsaturated aliphatic hydrocarbon alcohols containing 4 to 30 carbon atoms, or of aromatic alcohols (more particularly those for which the aromatic ring has 6 carbon atoms) possibly containing one or more substituents on the aromatic ring, selected from the alkyl and alkenyl radicals containing up to 30 carbon atoms, or selected from the alkaryl radicals for which the alkyl part, linear or non-linear, contains 1 to 10 carbon atoms and the aryl part contains 6 carbon atoms; the counter-ion is more particularly selected from the alkali metals, such as sodium and potassium, the ammonium ions of type NR₄+ with R, identical or different, representing a hydrogen atom, an alkyl radical containing 1 to 10 carbon atoms, preferably 1 to 4, possibly bearing a hydroxyl radical. Among the biological activators that can be envisaged, we may further mention mineral salts soluble in the aqueous phase, for example the salts of alkali metals, such as sodium and potassium, or of ammonium selected from the carbonates; the bicarbonates; the halides, such as chlorides and fluorides; the nitrates, the phosphates; the hydrogenphosphates; the sulfates; the bisulfates; the bisulfites, individually or mixed. The activators that have just been mentioned can be used individually or mixed.

When the dispersion according to the invention contains additives of this type, their content can represent up to 40 wt. % of the active substance or substances in the form of soluble salt (in other words, of the active substance or substances that is/are to be activated).

The dispersion according to the invention can in addition contain at least one thickening agent, for example hydrocolloids such as xanthan gum, galactomannans such as guar, carob; or their mixtures.

In the case when the dispersion contains at least one thickening agent, the total content of said compound is generally between 0.5 and 2 wt. % of the dispersion.

The dispersion according to the invention can also contain, advantageously, at least one biocidal agent (for example: proxel, glokill, etc.). If such a compound is used, its content is usually less than or equal to 0.5 wt. % of the dispersion, preferably about 0.1 wt. %.

An anti-gelling agent can also be included in the composition of the dispersion according to the invention, such as ethyleneglycol, propyleneglycol for example. Usually the content of anti-gelling agent, if present, is less than or equal to 10 wt. % relative to the weight of the dispersion; preferably between 2 and 10% of the aqueous phase of the dispersion.

Antifoaming agents can also be mentioned as additives that it is possible to use in plant protection formulations. The conventional compounds include silicones, and soaps of fatty acids, such as aluminum stearate for example.

If such compounds are used, they generally represent a content between 0.1 and 6 wt. % of the dispersion, preferably about 0.5 to 2 wt. % of the dispersion.

According to a variant of the present invention, the dispersion can be in a dry form.

By dry form we mean more precisely compositions whose residual water content is such that particles are obtained whose appearance is that of a non-sticky, non-pasty powder.

The invention also relates to a method of preparation of a dispersion of said kind in which an aqueous solution of copolymer and of soluble salt is prepared, to which the organic phase, possibly containing an active substance, is added progressively.

In the case of a dispersion containing a solid dispersed in the aqueous phase, said solid is preferably added to the emulsion once the latter is obtained.

It should be noted that it may be appropriate to proceed to a step of pre-grinding of the solid before it is introduced into the emulsion.

In the case when the dispersion contains additives, the additives that are soluble in the aqueous phase are added to the latter before adding the organic phase; the additives that are soluble in the organic phase are added to the latter before said organic phase is added to the aqueous phase.

Said preparation is carried out with stirring. It should be noted that equipment of the Ultra-Turrax type is appropriate for this.

The temperature of preparation of the dispersion can vary over a wide range as soon as the organic phase is in the liquid state during the preparation of the emulsion. Usually, however, the temperature of preparation of the dispersion is between 15 and 40° C.

The stirring time can be determined without difficulty by a person skilled in the art. One possible criterion for this is, for example, the time taken to obtain an emulsion with a suitable average droplet size.

In the case when the dispersion is in a dry form, a drying step is carried out once the dispersion is obtained.

In this scenario, it may be advantageous to add an additional quantity of copolymer to the dispersion once obtained, in such a way that the total content of the copolymer, in dry weight, is between 10 and 20 wt. % relative to the organic phase.

According to a first embodiment of the invention, drying in a stove can be envisaged. Preferably, this drying takes place in a thin layer.

Usually the drying temperature is less than or equal to 100° C. More particularly, temperatures between 50 and 90° C. are suitable for application of this method.

According to another particular embodiment of the invention, the dispersion is dried by spraying, by lyophilization (freezing-sublimation), or using a Duprat® drum.

These forms of drying, and notably spray-drying, are particularly indicated because they enable the condition of the emulsion to be preserved and granules to be directly obtained.

Spray-drying can be effected in the usual way in any known equipment, for example a spraying tower that combines spraying by means of a nozzle or a turbine with a stream of hot gas.

The inlet temperature of the hot gas (generally air), at the top of the column, is preferably between 100 and 115° C. and the outlet temperature is preferably between 55 and 65° C. These temperatures are given as a guide, and depend on the thermal stability of the various components.

In the case of operations of emulsion drying carried out using the Duprat® drum, or using any means for fast production of a dry film, which is separated from the drying support by a scraping operation for example, particles are obtained which can be ground if required. If necessary, these particles can be processed further, such as in an agglomeration step, so as to obtain granules.

Finally, the present invention relates to the use in the area of plant protection formulations.

Concrete but non-limiting examples of the invention will now be presented.

### EXAMPLES 1

These examples relate to the production of diblock polymers.

In these examples, the number-based molar masses were evaluated on the monoblock (prepolymer) and on the copolymer, by GPC analysis (aqueous eluent) after sodium hydroxide hydrolysis. The values obtained by GPC are close to those expected for a hydrolyzed monoblock and a hydrolyzed block copolymer.

The values of the number-based molar masses shown are theoretical values calculated from the quantities of monomer and of control agent introduced. Controlled radical polymerization.

### Example 1a

### Synthesis of P1

p(ABu)₂₀₀₀-b-p (AM)₃₀₀₀ polymer with weight ratio

p(ABu)/p(AM):(40/60)

A) Step 1: Synthesis of the Monoblock p(ABu)₂₀₀₀

Composition of the Reaction Mixture:

| | | |
|---|---|---|
| Tetrahydrofuran | 30.00 | g |
| Butyl acrylate | 16.00 | g |
| S-ethylpropionyl O- | 1.664 | g |
| ethyldithiocarbonate | | |
| AIBN (azobisisobutyronitrile) | 0.263 | g |

The above ingredients are loaded in a 250-ml polymerization reactor equipped with a magnetic stirrer. The reaction is carried out under an atmosphere of dry nitrogen for 20 minutes, the reaction mixture is then heated to 60° C. and held at this temperature for 20 hours. Small quantities of samples of polymers are taken from time to time to monitor the conversion. The solids content is 30.2% at the end of the reaction.

B) Step 2: Synthesis of the Diblock p(ABu)₂₀₀₀-b-p(AM)₃₀₀₀

Composition of the Reaction Mixture:

| | | |
|---|---|---|
| Tetrahydrofuran | 100.00 | g |
| Acrylamide | 24.00 | g |
| AIBN (azobisisobutyronitrile) | 0.263 | g |

The above ingredients are loaded in a dry container under an atmosphere of dry nitrogen for 20 minutes, then transferred to the polymerization reactor, containing the polymer resulting from the preceding step (16 g), using a double-tip syringe. At the end of transfer, the reaction mixture is then heated to 60° C. and held at this temperature for 20 hours. Small quantities of samples of polymers are taken from time to time to monitor the conversion. The solids content is 30% at the end of the reaction.

The reaction mixture is left to cool and the solvents are eliminated using a rotary evaporator.

The number-based molar mass of the copolymer is 5000 g/mol (theoretical value).

### Example 1b

### Synthesis of P2

p(ABu)₁₀₀₀-b-p(AA)₄₀₀₀ polymer with weight ratio

p(ABu)/p(AA):(20/80)

A) Step 1: Synthesis of the Monoblock p(ABu)₁₀₀₀

The procedure of step 1) of example 1a is repeated exactly, with the following composition of the reaction mixture:

| | | |
|---|---|---|
| Ethanol | 23.00 | g |
| Butyl acrylate | 8.00 | g |
| S-ethylpropionyl O- | 1.664 | g |
| ethyldithiocarbonate | | |
| AIBN (azobisisobutyronitrile) | 0.263 | g |

The solids content is 30.2% at the end of the reaction.

B) Step 2: Synthesis of the Diblock p(ABu)₁₀₀₀-b-p(AA)₄₀₀₀

The procedure of step 2) of example 1a is repeated, starting from 8 g of the product prepared previously in step 1, with the following composition of the reaction mixture:

| | | |
|---|---|---|
| Ethanol | 93.00 | g |
| Acrylic acid | 32.00 | g |
| AIBN (azobisisobutyronitrile) | 0.263 | g |

The reaction mixture is left to cool and the solvents are eliminated using a rotary evaporator.

The solids content is 30% at the end of the reaction.

The number-based molar mass of the copolymer is 5000 g/mol (theoretical value).

### Example 1c

### Synthesis of P3

p(ABu)₂₀₀₀-b-p(AA)₃₀₀₀ polymer with weight ratio

p(ABu)/p(AA):(40/60)

1) Step 1: Synthesis of the Monoblock p(ABu)₂₀₀₀

Composition of the Reaction Mixture:

| | | |
|---|---|---|
| Ethanol | 39.50 | g |
| Butyl acrylate | 16.00 | g |
| S-ethylpropionyl O- | 1.664 | g |
| ethyldithiocarbonate | | |
| AIBN (azobisisobutyronitrile) | 0.263 | g |

The above ingredients are loaded in a 250-ml polymerization reactor equipped with a magnetic stirrer.

The reaction is carried out under an atmosphere of dry nitrogen for 20 minutes, the reaction mixture is then heated to 60° C. and held at this temperature for 20 hours.

Small quantities of samples of polymers are taken from time to time to monitor the conversion.

The solids content is 30%.

2) Step 2: Synthesis of the Diblock p(ABu)₂₀₀₀-b-p(AA)₃₀₀₀

Composition of the reaction mixture, added to 16 g of precursor polymer obtained in the preceding step 1:

| | | |
|---|---|---|
| Ethanol | 93.00 | g for 30% of solid |
| | | at the end |
| Acrylic acid | 24.00 | g |
| AIBN | 0.066 | g |
| (azobisisobutyronitrile) | | |

The above ingredients are loaded in a dry container under an atmosphere of dry nitrogen for 20 minutes, then transferred to the polymerization reactor using a double-tip syringe.

At the end of transfer, the reaction mixture is then heated to 60° C. and held at this temperature for 20 hours.

Small quantities of samples of polymers are taken from time to time to monitor the conversion.

The solids content is 30%.

The reaction mixture is left to cool and the solvents are eliminated almost completely using a rotary evaporator.

The number-based molar mass of the copolymer is 5000 g/mol (theoretical value).

### Example 1d

### Synthesis of P4

p(ABu)₇₅₀₀-b-p(AA)₇₅₀₀ polymer with weight ratio

p(ABu)/p(AA):(50/50)

The following mixture is introduced into a reactor equipped with a magnetic stirrer and a reflux column, and containing 160 g of acetone:

| | | |
|---|---|---|
| S-ethylpropionyl O- | 3.04 | g |
| ethyldithiocarbonate | | |
| Isopropanol | 21.24 | g |
| AIBN (azobisisobutyronitrile) | 0.82 | g |

The mixture is then stirred and kept under reflux at 70° C. 66 g of acrylic acid and 15 g of water are added gradually over the space of 3 hours. Then 0.41 g of azobisisobutyronitrile is added after an hour of addition then again 0.41 g of azobisisobutyronitrile is added after a second hour of addition. Once the addition of acrylic acid is completed, polymerization is allowed to proceed for one hour.

The temperature is then lowered to 65° C. by adding 560 g of acetone. While maintaining the temperature at 65° C., 140 g of butyl acrylate is added gradually over the space of 3 hours. 0.40 g of AIBN is added at the beginning of addition of the butyl acrylate. The reaction is allowed to proceed for a further 3 hours.

The reaction mixture is left to cool and the solvents are eliminated using a rotary evaporator. The residue obtained is dispersed in water and freeze-dried.

The number-based molar mass of the copolymer is 15000 g/mol (theoretical value).

### Example 1g

### Synthesis of P5

p(ABu)₁₀₀₀-b-p (AM)₄₀₀₀ polymer with weight ratio

p(ABu)/p(AM):(20/80)

1) Step 1: Synthesis of the Monoblock p(ABu)₁₀₀₀

The procedure of step 1) of example 1a is repeated, with the following composition of the reaction mixture:

| | | |
|---|---|---|
| Tetrahydrofuran | 23.00 | g |
| Butyl acrylate | 8.00 | g |
| S-ethylpropionyl O- | 1.664 | g |
| ethyldithiocarbonate | | |
| AIBN (azobisisobutyronitrile) | 0.263 | g |

The solids content is 30.2% at the end of the reaction.

2) Step 2: Synthesis of the Diblock p(ABu)₁₀₀₀-b-p(AM)₄₀₀₀

Step 2 of example 1a is repeated, with the following composition of the reaction mixture:

| | | |
|---|---|---|
| Tetrahydrofuran | 93.00 | g |
| Acrylamide | 32.00 | g |
| AIBN (azobisisobutyronitrile) | 0.263 | g |

The solids content is 30% at the end of the reaction.

The number-based molar mass of the copolymer is 5000 g/mol (theoretical value).

### EXAMPLE 2

This example relates to the preparation of emulsion containing 20% of organic phase (methyl ester of colza, Phytorob 926-65 sold by the company Novance) emulsified in an aqueous phase containing 55% of isopropylammonium salt of glyphosate (i.e. 3M with respect to salt).

The emulsion contains 2% of an ionic or nonionic amphiphilic diblock copolymer.

All the percentages given are percentages by weight.

Preparation of the Emulsion:

The quantity of copolymer required, dissolved beforehand at 20 wt. % in water, is added to the solution of glyphosate salt. The aqueous phase obtained is clear or slightly turbid but remains homogeneous, without any signs of macroscopic phase separation at rest, at room temperature.

The organic phase is added to this aqueous phase in 1 minute with mechanical stirring with a stirrer of the UltraTurrax type rotating at 8000 rpm. Then stirring of the emulsion obtained is continued for 2 minutes at a speed of 16000 rpm.

Properties of the Emulsion (Granulometry):

The granulometry of the emulsion thus obtained is measured 24 hours and 1 month after its preparation using a laser diffraction granulometer of the HORIBA type, and the median diameter is evaluated.

The median diameter (d₅₀) signifies that 50% of the particles, by volume, have a size less than or equal to the value of said diameter.

| | | Median |
|---|---|---|
| | Age of the | diameter |
| Polymer | emulsion | (μm) |
| P2 PABu-b-PAM | 24 h | 1.0 |
| 2000-b-3000 | 1 month | 1.0 |

PABu (polybutylacrylate)
PAM (polyacrylamide)

### EXAMPLE 3

This example relates to emulsions containing 20% of organic phase (colza methyl ester) emulsified in an aqueous phase containing 55% of isopropylammonium salt of glyphosate (i.e. 3M of salt).

The emulsion contains 1 or 2% of a PABu-PAA diblock copolymer in which the mass ratios are different. All percentages given are percentages by weight.

Preparation of the Emulsion:

The emulsion is prepared following the same procedure as that described in example 2.

Properties of the Emulsion (Granulometry):

The granulometry of the emulsion thus obtained is measured 24 hours and 1 month after its preparation using a laser diffraction granulometer of the HORIBA type.

| | | Median |
|---|---|---|
| | Age of the | diameter |
| Polymer | emulsion | (μm) |
| P2 2% PABu-b-PAA | 24 h | 0.9 |
| 1000-b-4000 | 1 month | 0.9 |
| P3 1% PABu-b-PAA | 24 h | 1.0 |
| 2000-b-3000 | 1 month | 1.2 |
| P4 2% PABu-b-PAA | 24 h | 1.2 |
| 7500-b-7500 | 1 month | 1.2 |

PAA (polyacrylic acid)

### EXAMPLE 4

This example relates to emulsions containing 20% of organic phase (colza methyl ester) emulsified in an aqueous phase containing 46.5% of isopropylammonium salt of glyphosate (i.e. 2.5M of salt).

The emulsion contains between 0.25 and 2% of an ionic or nonionic amphiphilic diblock copolymer (between 1.25 and 10 wt. % relative to the organic phase). All the percentages given are percentages by weight.

Preparation of the Emulsion:

The required quantity of polymer, previously dissolved at 20 wt. % in water, is added to the solution of glyphosate salt at the required concentration.

The aqueous phase obtained is clear or slightly turbid but remains homogeneous, showing no signs of macroscopic phase separation at rest.

The organic phase is added to said aqueous phase in 1 minute with mechanical stirring by a stirrer of the UltraTurrax type rotating at 8000 rpm.

Stirring of the emulsion obtained is continued in a MICROFLUIDICS M110S high-pressure homogenizer (homogenization pressure 500 bar).

Properties of the Emulsion (Granulometry):

The granulometry of the emulsion thus obtained is measured 1 hour and 15 days after its preparation, using a laser diffraction granulometer of the HORIBA type.

| | % | | |
|---|---|---|---|
| | copolymer/ | | Median |
| | organic | | diameter |
| Emulsion | phase | Time (days) | (μm) |
| P2 | 1.25 | 0 | 1.3 |
| PABu-PAA | | 15 | 1.5 |
| 1000-4000 | 2.5 | 0 | 0.8 |
| | | 15 | 0.9 |
| | 5 | 0 | 0.4 |
| | | 15 | 0.5 |
| | 10 | 0 | 0.4 |
| | | 15 | 0.5 |
| P5 | 1.25 | 0 | 1.7 |
| PABu-PAM | | 15 | 2.0 |
| 1000-4000 | 2.5 | 0 | 1.0 |
| | | 15 | 1.1 |
| | 5 | 0 | 0.7 |
| | | 15 | 0.7 |
| | 10 | 0 | 0.4 |
| | | 15 | 0.4 |

### EXAMPLE 5

The purpose of this example is to demonstrate the existence of synergy between the copolymer and the surfactant (alkylpolyglucoside).

Preparation of the Emulsion:

The emulsion is prepared following the same procedure as that described in example 2.

Properties of the Emulsion (Granulometry and Stability):

The granulometry of the emulsion thus obtained is measured 1 hour and 8 days after its preparation using a laser diffraction granulometer of the HORIBA type.

In addition, the granulometry of the dispersion is measured 24 hours after diluting it to 10% in tap water.

| | | Median |
|---|---|---|
| Composition of the | Age of the | diameter |
| emulsion | emulsion | (μm) |
| 5% Plantaren 2000 | 1 hour | 2.9 |
| | 8 days | 3.0 |
| Dilution to 10% | 24 hours | 3.4 |
| 2% P2 | 1 hour | 1.0 |
| PABu-b-PAM 2000-b- | 8 days | 1.0 |
| 3000 | | |
| Dilution to 10% | 24 hours | 1.0 |
| 1% Plantaren 2000 | 1 hour | 0.39 |
| 1% P2 | 8 days | 0.38 |
| PABu-b-PAM 2000-b- | | |
| 3000 | | |
| Dilution to 10% | 24 hours | 0.38 |

1. A dispersion comprising at least one emulsion of an organic phase in an aqueous phase; the aqueous phase having a content of soluble salt of at least 0.5 mol/l, characterized in that the aqueous phase contains at least one amphiphilic block copolymer, comprising at least one hydrophilic block and at least one hydrophobic block, selected from the copolymers:
(i) which do not cause macroscopic phase separation in solution at 1 wt. %, in a mixture of water and KBr; the concentration of KBr being identical to that of soluble salt of the dispersion, at 20° C.;
(ii) whose mass ratio of hydrophilic block(s) to hydrophobic block(s) is between 40/60 and 95/5.

2. The dispersion as claimed in the preceding claim, characterized in that the copolymer is selected from those whose mass ratio of hydrophilic block(s) to hydrophobic block(s) is between 50/50 and 90/10.

3. The dispersion as claimed in one of the preceding claims, characterized in that the hydrophilic blocks of the copolymer are obtained by employing, as hydrophilic monomer, at least one ionic or potentially ionizable hydrophilic monomer, more particularly anionic or cationic, and/or at least one nonionic hydrophilic monomer.

4. The dispersion as claimed in one of the preceding claims, characterized in that the anionic hydrophilic monomers contain at least one carboxylic, sulfonic, sulfuric, phosphonic, phosphoric, or sulfosuccinic function, or the corresponding salts.

5. The dispersion as claimed in the preceding claim, characterized in that the anionic hydrophilic monomers are selected from:
the linear, branched, cyclic or aromatic mono- or polycarboxylic acids, the N-substituted derivatives of acids, the monoesters of polycarboxylic acids, containing at least one ethylenic unsaturation;
the linear, branched, cyclic or aromatic vinylcarboxylic acids;
the amino acids containing one or more ethylenic unsaturations;
individually or mixed, their precursors, their sulfonic or phosphonic derivatives, as well as the macromonomers derived from said monomers; it being possible for the monomers or macromonomers to be in the form of salts.

6. The dispersion as claimed in one of the preceding claims, characterized in that the cationic hydrophilic monomers are selected from:
the aminoalkyl (meth)acrylates, the aminoalkyl (meth)acrylamides;
monomers containing at least one secondary, tertiary or quaternary amine function, or a heterocyclic group containing a nitrogen atom, vinylamine, ethyleneimine;
diallyldialkylammonium salts;
individually or mixed, or the corresponding salts of ammonium type having a counterion selected from the halides, the sulfates, the hydrosulfates, the alkylsulfates, the phosphates, the citrates, the formates, or the acetates; as well as the macromonomers derived from said monomers.

7. The dispersion as claimed in one of the preceding claims, characterized in that the nonionic hydrophilic monomers are selected from: ethylene oxide; the amides of linear, branched, cyclic or aromatic mono- or polycarboxylic acids, containing at least one ethylenic unsaturation or derivatives, such as (meth)acrylamide, N-methylol(meth)acrylamide; the hydrophilic esters derived from (meth)acrylic acid, for example 2-hydroxyethyl (meth)acrylate; vinyl esters by means of which it is possible to obtain polyvinyl alcohol blocks after hydrolysis, such as vinyl acetate, vinyl Versatate®, vinyl propionate; monomers of the sugar type such as osides, highly depolymerized polyholosides.

8. The dispersion as claimed in one of the preceding claims, characterized in that the hydrophilic monomers are selected from acrylic acid or its salts, acrylamide, 2-acrylamido-2-methylpropanesulfonic acid or its salts, and styrenesulfonic acid or its salts, vinylbenzyl trimethylammonium chloride, individually or in mixtures, or in the form of macromonomers.

9. The dispersion as claimed in one of the preceding claims, characterized in that at least one of the hydrophilic blocks is made up of one or more hydrophilic monomers or of a mixture of hydrophilic and hydrophobic monomer(s); the proportion by weight of hydrophobic monomer(s) representing more particularly less than 50% of the monomers forming said hydrophilic block, and preferably less than 20%.

10. The dispersion as claimed in one of the preceding claims, characterized in that the hydrophobic monomers are selected from:
the linear, branched, cyclic or aromatic esters of mono- or polycarboxylic acids, containing at least one ethylenic unsaturation,
αβ-ethylenically unsaturated nitrites, vinyl ethers, vinyl esters, vinylaromatic monomers, halides of vinyl or of vinylidene,
linear or branched, aromatic or non-aromatic hydrocarbon monomers, containing at least one ethylenic unsaturation,
individually or mixed, as well as the macromonomers derived from said monomers.

11. The dispersion as claimed in one of the preceding claims, characterized in that the hydrophobic monomers are selected from the esters of acrylic acid with linear or branched C₁-C₄ alcohols such as methyl, ethyl, propyl and butyl acrylate, the vinyl esters such as vinyl acetate, styrene, α-methylstyrene.

12. The dispersion as claimed in one of the preceding claims, characterized in that at least one of the hydrophobic blocks is made up of one or more hydrophobic monomers or of a mixture of hydrophilic and hydrophobic monomer(s); the proportion by weight of hydrophilic monomer(s) representing more particularly less than 50% of the monomers forming said hydrophobic block.

13. The dispersion as claimed in one of the preceding claims, characterized in that the copolymer is such that the number-based molar mass of each block is between 500 and 100·10³ g/mol, preferably between 10³ and 50·10³ g/mol.

14. The dispersion as claimed in one of the preceding claims, characterized in that the copolymers are of linear form.

15. The dispersion as claimed in one of the preceding claims, characterized in that the copolymers are diblocks.

16. The dispersion as claimed in one of the preceding claims, characterized in that the content of copolymer in the dispersion represents more particularly at least 1 wt. % relative to the organic phase, advantageously from 2 to 20 wt. % relative to the organic phase, and preferably between 2 and 10 wt. % relative to the organic phase.

17. The dispersion as claimed in one of the preceding claims, characterized in that the concentration of salt soluble in the aqueous phase is greater than 0.8 mol/l, advantageously greater than or equal to 1 mol/l, preferably greater than or equal to 2 mol/l.

18. The dispersion as claimed in one of the preceding claims, characterized in that the soluble salt is selected from the active substances that can be used in the area of plant protection.

19. The dispersion as claimed in claim 18, characterized in that the active substance is selected from glyphosate, sulfosate, glufosinate, in the form of alkali metal salts; in the form of ammonium salts, of type N(R)₄+ for which the R radicals, identical or different, represent a hydrogen atom or a linear or non-linear, saturated or unsaturated C₁-C₆ hydrocarbon radical, possibly substituted by a hydroxyl group; or alternatively in the form of sulfonium salts; said salts being present individually or in combination.

20. The dispersion as claimed in one of the preceding claims, characterized in that it comprises a solid dispersed in the aqueous phase.

21. The dispersion as claimed in one of the preceding claims, characterized in that the organic phase of the emulsion is selected from the organic compounds that are in a liquid form at the temperature of preparation of the dispersion, or alternatively from those that are liquid at the temperature of use of said dispersion, or even these two possibilities simultaneously.

22. The dispersion as claimed in the preceding claim, characterized in that the organic phase of the dispersion is selected from organic oils, waxes or fats of vegetable or animal origin; mineral oils or waxes; products from the alcoholysis of the aforementioned oils; saturated or unsaturated fatty acids; esters of said acids; saturated or unsaturated fatty alcohols; aliphatic or aromatic hydrocarbon solvents; petroleum fractions; individually or mixed.

23. The dispersion as claimed in one of the preceding claims, characterized in that the organic phase can contain one or more active substances that are in a form that is soluble in said organic phase or alternatively are dissolved in the latter, or it can even constitute an active substance itself.

24. The dispersion as claimed in one of the preceding claims, characterized in that the proportion by weight of organic phase in the dispersion represents 5 to 50 wt. %, preferably 10 to 30 wt. %.

25. The dispersion as claimed in one of the preceding claims, characterized in that the dispersion contains at least one surfactant that is soluble in the aqueous phase.

26. The dispersion as claimed in the preceding claim, characterized in that the surfactant is selected from the alkylpolyglucosides.

27. The dispersion as claimed in the preceding claim, characterized in that the content of surfactant represents at most 50 wt. % of the combined weight of surfactant+copolymer.

28. The dispersion as claimed in one of the preceding claims, characterized in that the aqueous phase of the dispersion contains a biological activator of the active substance or substances in the form of water-soluble salt.

29. The dispersion as claimed in the preceding claim, characterized in that the content of biological activator represents up to 40 wt. % of the active substance or substances.

30. The dispersion as claimed in one of the preceding claims, characterized in that the dispersion is in a dry form.

31. A method of preparation of a dispersion as claimed in one of the preceding claims, characterized in that an aqueous solution of copolymer, of soluble salt, is prepared, then the organic phase, possibly containing an active substance, is added progressively.

32. The method of preparation as claimed in the preceding claim, characterized in that the dispersed solid is added to the emulsion, once the latter is obtained.

33. The method of preparation as claimed in one of claims 31 or 32, characterized in that a drying step is carried out once the dispersion is obtained.

34. The method of preparation as claimed in the preceding claim, characterized in that an additional quantity of copolymer is added to the dispersion once obtained, in such a way that the total content of the copolymer, in dry weight, is between 10 and 20 wt. % relative to the organic phase.

35. The method of preparation as claimed in one of claims 33 or 34, characterized in that the drying is carried out in a stove, preferably in a thin layer; by spraying, by lyophilization (freezing-sublimation), or using a Duprat® drum.

36. The use of the dispersion as claimed in one of claims 1 to 30, or that can be obtained as claimed in one-of claims 31 to 35, in the area of plant protection formulations.
